(19) United States
(12) Patent Application Publication
(10) Pub. No.: US 2022/0032283 A1
(43) Pub. Date: Feb. 3, 2022

(54) DISSOLUTION DEVICE

(71) Applicant: KLARIA PHARMA HOLDING AB, Uppsala (SE)

(72) Inventors: Blaz GRILC, Uppsala (SE); Odon PLANINSEK, Uppsala (SE); Scott BOYER, Uppsala (SE); Fredrik HUBINETTE, Uppsala (SE); Erik MASCHER, Uppsala (SE)

(73) Assignee: KLARIA PHARMA HOLDING AB, Uppsala (SE)

(21) Appl. No.: 17/277,515

(22) PCT Filed: Sep. 10, 2019
(86) PCT No.: PCT/EP2019/074127

§ 371 Date: Mar. 18, 2021

(30) Foreign Application Priority Data
Sep. 18, 2018 (GB) 1815201.7

(51) Int. Cl. B01L 3/00; G01N 13/00; G01N 33/15

(52) U.S. Cl. CPC B01L 3/502; G01N 13/00; G01N 33/15; B01L 2200/026; B01L 2200/0605; G01N 2013/006; B01L 2200/025; B01L 2300/0609; B01L 2300/0663; B01L 2300/14; B01L 2400/0457; B01L 2200/143

(57) ABSTRACT

A dissolution device for measuring a dissolution rate of a test sample in a fluid, the device comprising a first cavity and a second cavity, wherein each of the first and second cavities has a fluid inlet for connection to a fluid supply, and wherein the device comprises an opening between the first and second cavities, and the device comprises a sample support configured to position the test sample across the opening.

### FIELD OF INVENTION

The present invention relates to the field of testing pharmacological compositions, in particular the field of dissolution testing.

### BACKGROUND

American and European pharmacopoeias describe several different methods for testing the dissolution of oral dosage forms. In particular, the United States Pharmacopeial Convention, Chapter 711, describes dissolution apparatus I, dissolution apparatus II, dissolution apparatus III and dissolution apparatus IV. For various dosage forms, these methods are suitable for testing the release of active substances, but there are also exceptions for which pharmacopoeia-recognised methods are not the preferred choice. These exceptions include oral dosage forms, for example oral films. These known methods do not give reproducible and discriminatory results and may therefore be unsuitable for film formulations, for example. Differences in release profiles between formulations are small and difficult to trace, even in formulations with different chemical composition.

A disadvantage of existing methods is that they require a large volume of dissolution medium and a thorough mixing of it. Such conditions are not representative of those of oral administration of oral dosage form in reality. Consequently, with existing methods, oral dosage formulations are dissolved substantially faster in testing than in the oral administration of the dosage form. The result of this may be the incorrect or misleading result of in-vitro testing of the investigated formulation.

Alternative methods for testing the dissolution of oral dosage forms, based on the use of a flow cell, are known. The flow cells generally comprise one chamber into which the investigated dosage form is inserted. Through the cell, a medium is pumped using a peristaltic pump or a piston (syringe) pump. The dosage form is then dissolved and an amount of the released active substance is monitored at an outlet of the cell.

U.S. Pat. No. 7,892,492 describes a flow cell for investigating the properties of solid dosage forms. The flow cell described by the patent has one chamber, and consequently the formulation is wetted only from one side of the dosage form, which may produce unreliable results because it is not representative of oral administration.

CN 2016/2,481,967 U describes a flow cell with a single chamber which uses a peristaltic pump to pump the medium. As explained above, flow cells with a single chamber may produce unreliable results. Furthermore, peristaltic pumps create a pulsating liquid flow which may adversely affect the dissolution of the pharmaceutical form.

The present invention seeks to provide an improved testing approach.

### SUMMARY

There is disclosed a device for measuring a dissolution rate of a test sample in a fluid, the device comprising: a first cavity; and a second cavity; wherein each of the first and second cavities has a fluid inlet for connection to a fluid supply; and wherein the device comprises an opening between the first and second cavities, and the device comprises a sample support configured to position the test sample across the opening.

There is also disclosed a method of measuring a dissolution rate of a test sample in a fluid, the method comprising the steps of: providing a first supply of fluid in a first cavity; providing a second supply of fluid in a second cavity, wherein an opening extends between the first and second cavities; and positioning the test sample across the opening.

### FIGURES

FIG. 1 shows a cross-sectional view of a flow cell.

FIG. 2 depicts a set-up for dissolution testing using a flow cell.

FIG. 3 depicts a set-up allowing adjustment of a vertical fluid level in a fluid container.

FIG. 4 shows the experimental results of an example dissolution process.

### DETAILED DESCRIPTION

An aspect of the present invention is directed to a dissolution device for measuring a dissolution rate of a test sample in a fluid. The device comprises a first cavity and a second cavity. The first and second cavities each have a fluid inlet for connection to a fluid supply. The device comprises an opening between the first and second cavities. A sample support is provided to position a test sample across the opening.

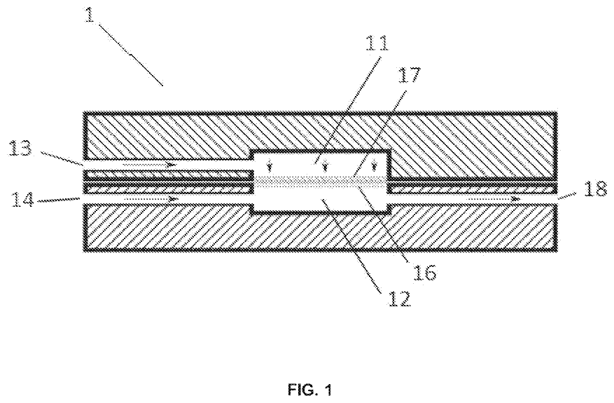

FIG. 1 shows an example of a flow cell 1 which is in accordance with the dissolution device disclosed herein. The flow cell 1 may be a single component or may be an assembly of two or more components. In an arrangement, one component may be shaped to provide a first cavity 11 and a fluid inlet 13, and another component may be shaped to provide a second cavity 12 and a fluid inlet 14. The first and second cavities 11, 12 may have the same shape or same size or same volume, or may have different shapes, sizes or volumes.

As shown in the figure, each component may be in a block shape. The two components may be fixed together by bolts so that a water-tight contact is formed between the two components. Each of the two components may comprise a cut-out on the side in contact with the other of the two components. The cut-outs may be shaped and positioned on the components so that, when the two components are assembled together, the cut-outs align and form an opening between the first cavity 11 and the second cavity 12. It is understood that the opening may be of any shape or size as may be required.

A sample support 16 may be provided, which may position the test sample 17 across the opening. The sample support 16 may provide mechanical support to the test sample. In an arrangement, the sample support 16 may be permeable. For example, the sample support 16 may be a permeable membrane, a mesh, a perforated plate, a fibrous sheet, a foam sheet, a dialysis membrane, an osmotic active membrane, a filter, any porous hydrophobic or hydrophilic membrane, a sintered glass plate, a sintered metal plate, or the like. Although FIG. 1 shows that the test sample 17 is placed on the side of the sample support 16 facing the first cavity 11, the test sample 17 may alternatively be placed on the side of the sample support 16 facing the second cavity 12.

Depending on the physical or chemical form of the test sample 17, different materials may be used for the sample support 16. The sample support 16 may be made of a material comprising a hydrophilic or hydrophobic material. It may be of natural or synthetic origin. It should be noted that any common materials or combinations of materials may be used to produce the sample support as may be deemed appropriate by one of ordinary skill in the art.

It is understood that multiple sample supports may be provided, and the multiple sample supports may be identical or different (e.g. made of different materials). For example, the test sample may be sandwiched between two permeable membranes. For example, the test sample may be sandwiched between a permeable membrane on one side and a mesh on the other side.

The test sample 17 may be of any suitable shape or size. The test sample 17 may have the same size as the sample support 16 or have a different size. The test sample 17 may fully or partially cover the opening between the first and second cavities 11, 12.

It should be understood that different test samples may be advantageously used in the devices and methods herein disclosed, including solid, semi-solid and trans-dermal pharmaceutical forms. It should also be noted that the test sample 17 may be of any shape or form as may be required, including powders, crystals, pastes, gels, emulsions, solid self-emulsifying systems, colloids, tablets, pellets, granules, capsules, microcapsules, nanofibers, impregnated pads, orodispersible formulations, oral dosage forms and oral films. The disclosed devices and methods may be particularly effective for testing oral dosage forms, especially oral films. As is generally known, oral dosage forms are formulations that release the whole active substance in most cases (typically from a few seconds to a maximum of about 10 minutes).

The fluid inlet 13 of the first cavity may be connected to a first fluid supply, and the fluid inlet 14 of the second cavity may connected to a second fluid supply. The first and second cavities 11, 12 may be in fluid communication respectively with the first and second fluid supplies via the respective fluid inlets. The first and second cavities 11, 12 may be filled with fluid from the respective fluid supply.

It is understood that the fluid inlets 13, 14 may be connectable to the fluid supplies using any kind of connection. For example, the fluid inlet 13, 14 may be a simple cylindrical bore, and the fluid supply may be a flexible tube sized for insertion into the bore using an interference fit. The fluid inlet 13, 14 may alternatively comprise a spout, and the fluid may be a flexible tube sized to accept the spout. The connection may alternatively be a screw-type connector. Any other known kinds of connection may be used.

The test sample 17 may be wetted by fluid from the first and second fluid supplies and may be allowed to dissolve over a period of time. A high concentration of dissolved test sample may form locally on and near the surface of the undissolved portion of the test sample 17. Dissolved test sample in the first cavity 11 may traverse the opening between the first and second cavities 11, 12. For example, dissolved test sample on the surface of the test sample 17 facing the first cavity 11 may diffuse through the test sample 17 and the sample support 16 and reach the second cavity 12. Where the test sample 17 does not cover the opening entirely, some or all of the dissolved test sample on the surface of the test sample 17 facing the first cavity 11 may alternatively or additionally reach the second cavity 12 through a portion of the opening that is not covered by the test sample 17 and through the sample support 16.

The static pressure of the fluid in the first cavity 11, measurable at a point within the first cavity and adjacent the test sample, may be equal to or higher than the static pressure of the fluid in the second cavity 12, measurable at a point within the second cavity and adjacent the test sample. A relative pressure may thus exist between the first and second cavities 11, 12. The relative pressure may have the effect of facilitating the passing of the dissolved test sample from the first cavity 11 to the second cavity 12. The relative pressure may also prevent the fluid in the second cavity 12 from passing into the first cavity 11.

The relative pressure between the first and second cavities may be chosen from an appropriate range, e.g. 0-20,000 Pa, optionally approximately 400-1,500 Pa. This may be achieved by providing fluid supplies that are appropriately configured.

It may be desirable to ensure that the fluid supplies are free of pressure pulsation as far as possible because pulsation in the fluid may adversely affect the dissolution of the test sample. For example, peristaltic pumps and piston pumps are known to produce pressure pulsation. Any known means of providing a static pressure without inducing pressure pulsation in the fluid may be used with the devices and methods disclosed herein.

The relative pressure between the first and second cavities 11, 12 may be substantially constant during some or all of the period of time when measurements are taken. Such an arrangement may help achieve controlled dissolution of the test sample 17. A substantially constant relative pressure may be achieved using any known means depending on the chosen means of providing static pressure.

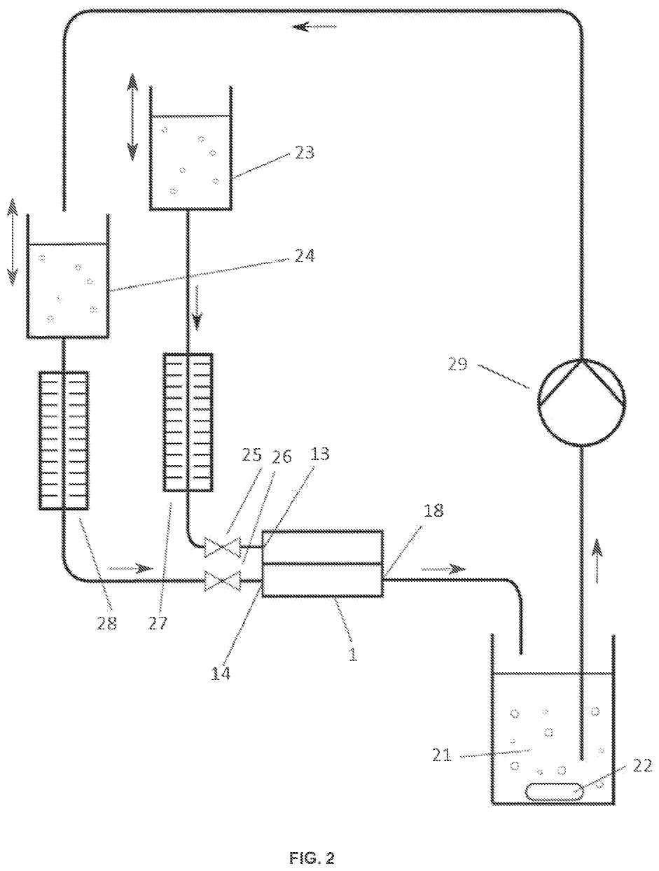

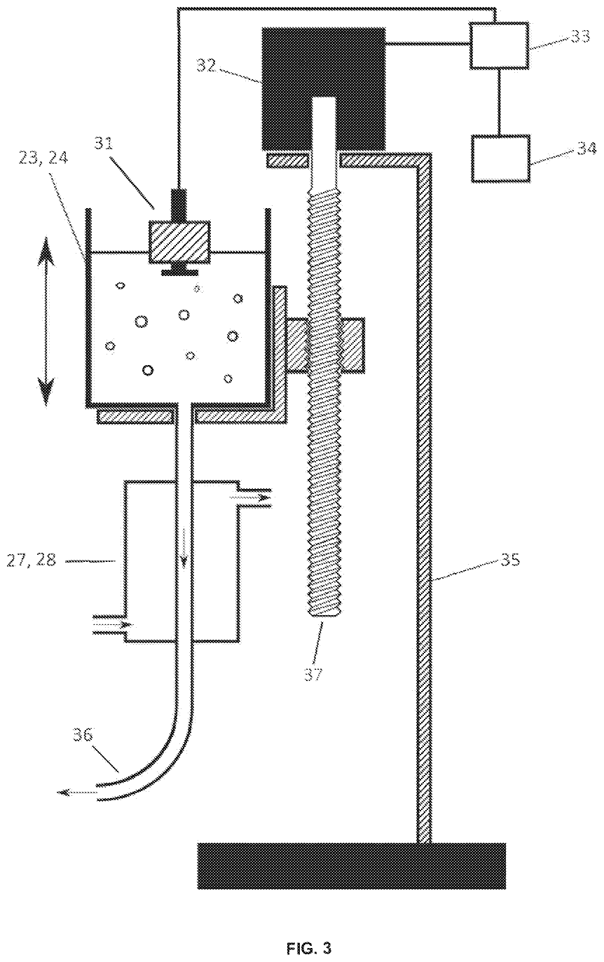

One particularly effective way of providing a static pressure in a fluid supply is to feed the fluid supply by gravity, thereby inducing a hydrostatic pressure. This may be achieved by providing a fluid container that feeds fluid into the fluid supply by gravity. This arrangement may be used for one or both of the first and second fluid supplies. As shown in FIGS. 2 and 3, the first and/or second fluid containers 23, 24 may be positioned at a vertical position that is higher than the flow cell 1. One or more of the fluid containers 23, 24 may be connected at the base to fluid inlet 13 or 14 via a tube 36.

Where the static pressure of one or both of the first and second fluid supplies is provided by a hydrostatic pressure, a vertical fluid level of the fluid supply, measured relative to a reference height, may be controlled. In addition, the vertical fluid level may be monitored manually or with the aid of one or more sensors. The vertical fluid level of a fluid supply may be controlled based on the manual monitoring or sensor output. The control of the vertical fluid level may be achieved manually or automatically.

With reference to the arrangement shown in FIGS. 2 and 3, the static pressure may be provided by a hydrostatic pressure induced by positioning each of the fluid containers 23, 24 at a vertical position. The relative pressure between the first and second fluid supplies may be provided primarily by the difference in vertical fluid level between the first and second fluid containers 23, 24. The relative pressure may be controlled by controlling the vertical position of one or both of containers 23, 24.

During the dissolution process, the fluid flows out of the containers 23, 24 into the flow cell 1 and, unless compensatory measures are taken, the vertical fluid level in the containers 23, 24 would decrease, leading to a decrease in the pressures of the fluid supplies and a change in the relative pressure between the first and second fluid supplies. Therefore, where the dissolution process is to be carried out under a substantially constant relative pressure, the pressure changes may be compensated for by adjusting the vertical fluid level in one or both of containers 23, 24 relative to the position of the flow cell 1.

FIG. 3 shows a fluid level adjuster which may be used to adjust the vertical fluid level in the fluid containers 23, 24. By maintaining a substantially constant height difference between the vertical fluid levels in the fluid containers 23, 24, a substantially constant relative pressure between the first and second fluid supplies may be obtained.

As shown in the figure, the container 23, 24, containing a quantity of fluid, may be placed on a support. The support may be movable by a linear actuator 37, such as a screw-type linear actuator, a pneumatic linear actuator, a hydraulic linear actuator, or the like. The linear actuator 37 may be driven by a motor 32, such as a stepper motor, a servo motor, a linear motor, or the like. By driving the linear actuator 37, the container 23, 24 may be shifted up or down as required. The container 23, 24, which may be movable by the linear actuator 37 via the support, may move up or down as a result of the change in vertical position of the linear actuator. The fluid in the container 23, 24 may follow the movement of the container 23, 24, and thereby the vertical fluid level in the container 23, 24 may be adjusted.

Furthermore, a fluid level sensor 31 may be provided to detect a change in the vertical fluid level in the container 23, 24. The fluid level sensor 31 may be a fixed height relative to the flow cell 1. The fluid level sensor 31 may be in contact with the fluid, or may be not in contact with the fluid. The fluid level sensor 31 may measure a fluid level in the container 23, 24. The fluid level sensor 31 may output data to a microcontroller 33. The microcontroller 33 may process the data and drive the motor 32 appropriately, thereby changing a vertical position of the linear actuator 37. The vertical fluid level may thus be controlled automatically.

The microcontroller 33 may further output the data to a computer 34, which may record movements of the motor 32 at a particular time.

The fluid level adjuster may employ negative feedback so as to regulate the vertical fluid level in the container 23, 24. Using negative feedback, the fluid level adjuster may be able to move the vertical fluid level up when the sensor 31 detects that the vertical fluid level is too low. Conversely, the fluid level adjuster may be able to move the vertical fluid level down when the sensor 31 detects that the vertical fluid level is too high. The vertical fluid level may thus be regulated at an appropriate level at all times.

Where both the first and second fluid containers 23, 24 are each provided with a fluid level adjuster, the fluid level adjusters may be independently controlled, thereby achieving independent adjustment of the vertical fluid level in each container 23, 24.

Alternatively, only one fluid container 23, 24 may be provided with a fluid level adjuster. In this case, the fluid level adjuster may adjust the vertical fluid level in said container so as to compensate for the change in vertical fluid level in both containers 23 and 24, thereby achieving control of the relative pressure between the fluids in the first and second cavities 11, 12.

Instead of or in addition to using the fluid level adjuster described above, a recirculation pump 29 may be used to recirculate fluid received from the flow cell 1 back into the fluid supply. As shown in FIG. 2, fluid in the second cavity 12 may drain out via an outlet 18. A reservoir 21 may be provided to collect the fluid draining from the second cavity 12. The fluid may then be transported by the recirculation pump 29 back into the second fluid container 24. This may have the effect of regulating the vertical fluid level in the second fluid container 24. Fluid may alternatively or additionally be transported in the same way into the first fluid container 23 as required.

In addition to collecting fluid draining from the second cavity 12, the reservoir 21 may also serve to provide a convenient means of taking fluid samples. A stirrer 22 may also be provided in the reservoir 21 to continuously stir the fluid within. This may ensure homogeneity of the fluid.

The recirculation pump 29 may be operable manually or automatically. A fluid level sensor 31, such as described above, may be used to detect a change in the vertical fluid level in the fluid container 23, 24, and the output of the sensor may be used for controlling the recirculation pump 29. The recirculation pump 29 may be operable at a fixed flow rate, or at two or more flow rates, or may be capable of continuously variable flow rate. As explained above, a microcontroller 33 may be provided to detect whether the fluid level in the container 23, 24 has fallen below a limit (or has emptied), and may switch on the recirculation pump 29 to pump fluid into the container 23, 24. The flow rate of the recirculation pump 29 may be chosen or controlled so as not to cause abrupt changes in the vertical fluid level in the container 23, 24. The flow rate of the recirculation pump 29 may be controlled so as to achieve a required vertical fluid level in the container 23, 24. The vertical fluid level in the container 23, 24 may be kept constant by operating the recirculation pump 29 at the flow rate at which fluid flows out of the container 23, 24.

The vertical fluid level in the containers 23, 24 may be controlled and adjusted using both the fluid level adjuster and the recirculation pump as described above. In such an arrangement, the recirculation pump 29 may pump fluid into the container 23, 24 as or when the fluid level falls, while at the same time the fluid level adjuster may adjust the vertical position of the container 23, 24 so as to ensure that vertical fluid level in the container 23, 24 remains at a constant height relative to the flow cell 1. The hydrostatic pressure induced in the fluid supply may thus be maintained.

The first cavity 11 may be configured to allow fluid to flow out only through the opening. It is understood that the first cavity 11 may comprise any arbitrary number of holes, apertures, cut-outs, and the like, so long as fluid is allowed to flow out of the first cavity 11 only through the opening between the first and second cavities 11, 12. For example, for manufacturing efficiency, the flow cell 1 may be formed of two identical components, each having an inlet, an outlet, and a cut-out to form the opening between the first and second cavities 11, 12 when the two components are assembled together, and the outlet in the component which is to be used as the component containing the first cavity 11 may be plugged so as to prevent fluid in the first cavity 11 from flowing out other than through the opening. Equally, any additional holes, apertures or cut-outs in the first cavity 11 may be used as additional fluid inlets.

The above arrangement may have the effect of maintaining the static pressure in the first cavity 11. This may be because the fluid flow rate in the first fluid supply may be kept relatively low. The fluid flow rate may be low because it is limited by the extent to which the test sample 17 and/or the sample support 16 are permeable. As the fluid flow rate is low, the flow velocity may be kept low and, by Bernoulli's principle, little of the static pressure in the first fluid supply is converted into kinetic energy. Thus, the static pressure of the fluid measurable in the first cavity adjacent the test sample 17 may be kept relatively high, and a relative pressure across the opening may be maintained. As explained above, the relative pressure may facilitate the passing of the dissolved test sample from the first cavity 11 into the second cavity 12.

It may also be desirable to control the temperature of the fluids during the dissolution process. With reference to FIGS. 2 and 3, one or both of the first and second fluid supplies may be equipped with a heat exchanger 27, 28 so as to regulate the temperature of the fluid before it enters the flow cell 1. The heat exchanger 27, 28 may heat or cool the fluid to a suitable temperature for use in the dissolution test, such as between 2° C. and 60° C., preferably about 37±1° C. Depending on the results obtained by the measurements, the temperature of the heat exchanger 27, 28 may be adjusted. The heat exchanger 27, 28 may be of any suitable type, such as water-filled, oil-filled, electric, induction, and the like.

A temperature sensor may be provided to monitor the temperature of the fluid. In an arrangement, the flow cell 1 may comprise a temperature sensor. It should be understood that a temperature sensor may be provided at any suitable location, including at the outlet 18 of the second cavity, or in a container collecting fluid flowing out through the outlet 18. A temperature sensor may be provided at a location where temperature measurements taken therein may be used to infer a temperature within the flow cell 1. The temperature sensor may be of any suitable type, including thermistor-type sensors, resistance-type sensors, thermocouples, semiconductor-type sensors, infrared sensors and other contact or non-contact temperature sensors.

Valves 25, 26 may be provided before the inlet to one or both of the first and second cavities 11, 12 so as to allow fluid flow to be stopped as required.

Another aspect of the present invention is directed to a method for measuring a dissolution rate of a test sample in a fluid. The method comprises the steps of providing a first supply of fluid in a first cavity; providing a second supply of fluid in a second cavity, wherein an opening extends between the first and second cavities; and positioning a test sample across the opening.

Any device disclosed herein may be used to carry out the above method. FIG. 1 shows one possible arrangement.

In preparation for the dissolution process, the two components may first be separated. A test sample 17 may be placed across the opening of the first cavity 11. At this point, the test sample 17 may be prevented from falling into the first cavity 11 by being supported by the periphery of the opening. A sample support 16 may be laid over the test sample 17. The component defining the second cavity 12 may be fitted onto the other component. This may fix the test sample 17 and the sample holder 16 firmly together.

Alternatively, after separating the two components, the sample support 16 may be placed on top of the second cavity 12 and across the opening. At this point, the sample support 16 may be prevented from falling into the second cavity 12 by being supported by the periphery of the opening. A test sample 17 may then be placed on top of the sample support 16. The component defining the first cavity 11 may be fitted onto the other component. This may fix the test sample 17 and the sample holder 16 firmly together.

The two components may be fixed together by bolts so that the contact between the components is watertight. If further sample supports are to be used, then the preparation may include installing further sample supports before installing the test sample 17, or may include placing further sample supports onto the one laid over the test sample 17.

The first supply of fluid may be provided by connecting the fluid inlet 13 of the first cavity to a first fluid supply, and the second supply of fluid may be provided by connecting the fluid inlet 14 of the second cavity to a second fluid supply. The first and second cavities 11, 12 may be filled with fluid from the respective fluid supply.

Depending on the chemical or physical properties of the test sample, different fluids may be supplied to the first and second cavities. Any suitable fluids may be used. Examples of fluids include: water, saline solutions, phosphate buffer pH 6.8, citrate buffer, MacIlvaine buffer, FaSSIF, FeSSIF, various recipes of simulated saliva, and the 2nd fluid from the Japanese Pharmacopeia (JP). A suitable fluid may be bio-relevant or bio-non-relevant. A suitable fluid may be relevant only in its pH value. The fluid supplied to each of the first and second cavities may have the same composition or may have different compositions. Prior to use, the fluid may be degassed so as to remove dissolved gasses from the fluid. This may prevent the formation of gas bubbles during the dissolution process.

It may be desirable to remove air from the dissolution device. In preparation for the dissolution process, the flow cell 1 may be empty and may be placed in a vertical position so that inlets 13 and 14 are respectively at the highest point of the first and second cavities 11, 12. Next, the first and second cavities 11, 12 may be filled with fluid. The fluid may displace air in the first and second cavities, and the air may float to the highest open liquid surface, whereupon the air leaves the fluid and passes to the atmosphere. All air bubbles may be removed from the system because they could inhibit the dissolution process and affect the flow or pressure of the fluid in the flow cell 1. When the flow cell 1 is full of fluid and free of air bubbles, it may be placed in a horizontal position such that the first cavity 11 is above the second cavity 12.

The test sample 17 may be wetted by fluid from the first and second fluid supplies and may be allowed to dissolve over a period of time. A high concentration of dissolved test sample may form locally on and near the surface of the undissolved portion of the test sample 17. Dissolved test sample in the first cavity 11 may traverse the opening between the first and second cavities 11, 12. For example, dissolved test sample on the surface of the test sample 17 facing the first cavity 11 may diffuse through the test sample 17 and the sample support 16 and reach the second cavity 12. Where the test sample 17 does not cover the opening entirely, some or all of the dissolved test sample on the surface of the test sample 17 facing the first cavity 11 may alternatively or additionally reach the second cavity 12 through a portion of the opening that is not covered by the test sample 17 and through the sample support 16.

The dissolved test sample may mix with the fluid in the second cavity 12. During the dissolution process, the concentration of the dissolved test sample may be measured. Measurements may be taken at regular intervals or irregular intervals or continuously, and may be taken manually or with an automatic sampler. Various methods of measurement are possible. For example, concentration may be measured at a point within the second cavity 12. Alternatively, fluid may be extracted from the second cavity 12, whereupon concentration measurements may be taken from the extracted fluid. The concentration of dissolved test sample within the second cavity 12 may be inferred from measurements taken from the extracted fluid. Fluid extracted from the second cavity 12 may be collected in a reservoir 21 to facilitate measurement. Fluid may be extracted at regular intervals or irregular intervals or continuously. For example, the second cavity 12 may be provided with an outlet 18, through which fluid may be extracted.

The use of the dissolution device as described above may provide test conditions that closely mimic the conditions of real-life oral administration. This may produce measurements that reliably indicate the quality of the test sample.

In an arrangement, as shown in FIG. 1, the fluid in the second cavity 12 may be allowed to flow out via the fluid outlet 18, carrying away any dissolved test sample. As fluid flows out of the second cavity 12, the cavity is replenished by the second fluid supply via the fluid inlet 14. The fluid may be allowed to flow continuously or discontinuously out of the second cavity 12. The flow out of the second cavity 12 may be controlled manually or automatically. With this arrangement, the dissolved test sample may be carried away so that the concentration of dissolved test sample in the second cavity 12 does not build up beyond a certain level.

A number of concentration measurements may be taken. The time at which each measurement is taken may be recorded. The initial point of dissolution may be when the fluid first wets the test sample 17. A data acquisition program may be initiated at that point to record measurements. The fluid may be analysed quantitatively. The measured concentration values and time records may be analysed to obtain a dissolution rate, a release profile, and the like. The results may be plotted in a chart. Preferably, the chart may be a plot of the released active pharmaceutical ingredient (API) as a percentage of the total amount in the test sample 17, vs. time. Alternatively or additionally, the chart may be a plot of concentration vs. time. In this way, a release profile of the active substance may obtained, which may serve for comparison between various formulations of test samples.

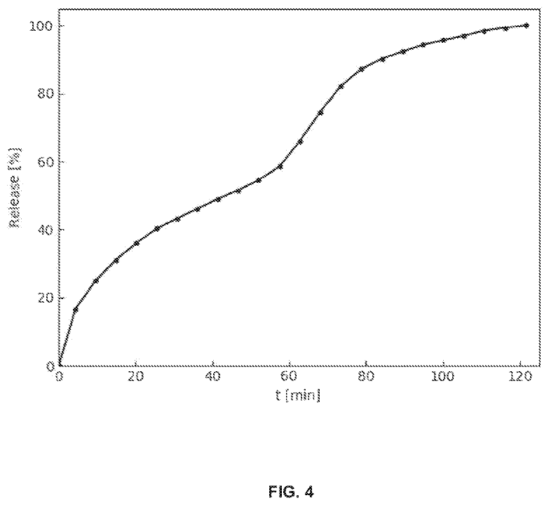

FIG. 4 shows the experimental results of a non-limiting example dissolution process. In this example, the first and second cavities both have a dimension of 18×28×5 mm. The temperature of fluid is controlled to 37±0.5° C. A relative pressure of approximately 1,000 Pa is maintained between the first and second cavities. 250 g of a 0.9% saline solution is used. An oral film is tested. At the start of the dissolution process, the oral film starts swelling from the top and bottom sides. At this time, the middle of the oral film is mostly intact and forms an impermeable barrier. This is detectable by a movement of fluid in the first fluid supply. In this example, the movement of fluid in the first fluid supply remains at 0 until the API release reaches 80%. From 0 to 20 min, a rapid start of the release is observed, which is followed by a slow decrease of the release rate. The release rate is approximately constant from 20 to 55 min (the chart shows a roughly linear portion during this period). The time length of this interval may be proportional to the thickness of the oral film. Then, at 55 min, the oral film swells sufficiently and becomes softer. The higher fluid pressure in the first cavity 11 forces the remainder of the oral film through the sample support 16 (which is a permeable membrane). At some point between 60 to 80 min (at around 80% API release), an opening is formed in the film so the fluid in the first cavity starts to bypass the test sample 17. As a result, a decrease in release rate is observable at around 80 min, and this lasts until the end of the dissolution process. Thus the dissolution process may be analysed as three phases: a swelling phase at 0-60 min, an extrusion phase at 60-80 min, and a final phase when the remainder of the test sample 17 is dissolved.

It should be noted that the above experimental results are for illustrative purposes only. Different experimental results may be obtained under different test parameters.

The static pressure of the fluid in the first cavity 11, measurable at a point within the first cavity and adjacent the test sample, may be set to be equal to or higher than the static pressure of the fluid in the second cavity 12, measurable at a point within the second cavity and adjacent the test sample. A relative pressure may thus exist between the first and second cavities 11, 12. The relative pressure may have the effect of facilitating the passing of the dissolved test sample from the first cavity 11 to the second cavity 12. The relative pressure may also prevent the fluid in the second cavity 12 from passing into the first cavity 11. As discussed above, the relative pressure between the first and second cavities may be chosen from an appropriate range, e.g. 0-20,000 Pa.

One particularly effective way of providing a relative pressure is to feed the fluid supply by gravity, thereby inducing a hydrostatic pressure. In this arrangement, this may be achieved by orienting the flow cell 1 such that the first cavity 11 on top of the second cavity 12, as shown in FIG. 1. This arrangement may induce a relative pressure between the first and second cavities 11, 12, measurable within the respective cavity and adjacent the test sample. This may cause the fluid in the first cavity 11, carrying dissolved test sample, to pass into the second cavity 12.

Alternatively or additionally, as discussed above, a relative pressure may be generated by providing a fluid container that feeds fluid into the fluid supply by gravity. This arrangement may be used for one or both of the first and second fluid supplies.

Where the static pressure of one or both of the first and second fluid supplies is provided by a hydrostatic pressure, a vertical fluid level of the fluid supply, measured relative to a reference height, may be controlled. In addition, the vertical fluid level may be monitored manually or with the aid of one or more sensors. The vertical fluid level of a fluid supply may be controlled based on the manual monitoring or sensor output. The control of the vertical fluid level may be achieved manually or automatically.

During the dissolution process, pressure changes in the fluid supplies may be compensated for by adjusting the vertical fluid level in one or both of containers 23, 24 relative to the position of the flow cell 1.

Any fluid level adjuster disclosed herein, such as that shown in FIG. 3, may be used to adjust the vertical fluid level in the fluid containers 23, 24. By maintaining a substantially constant height difference between the vertical fluid levels in the fluid containers 23, 24, a substantially constant relative pressure between the first and second fluid supplies may be obtained. The vertical fluid level may be controlled automatically.

The microcontroller 33 may further output the data to a computer 34, which may record movements of the motor 32 at a particular time.

Instead of or in addition to using the fluid level adjuster described above, fluid received from the flow cell 1 may be recirculated back into the fluid supply, such as by a recirculation pump 29 as described above.

The vertical fluid level in the containers 23, 24 may be controlled and adjusted using both the fluid level adjuster and the recirculation pump as described above.

Alternatively or additionally, the vertical fluid level in the container 23, 24 may be kept substantially constant by continuously adding fluid to the container at an excess rate so that the rate of addition of fluid is greater than the flow rate of fluid flowing from the container 23, 24 to the flow cell 1. In this arrangement, the container 23, 24 may be always full and any excess fluid added to the container 23, 24 may spill out at or near the top of the container 23, 24. In an arrangement, fluid may spill out at a top edge of the container 23, 24. In an arrangement, fluid may spill out via an outlet provided near the top of the container 23, 24. Any fluid that spills out may be captured and reintroduced into the fluid supply. In other words, the vertical fluid level may be controlled by setting the vertical position of a top edge or outlet of the container 23, 24. Any method of adding fluid may be used. Fluid may be added manually or automatically. In an arrangement, the recirculation pump 29 may be used to add fluid at an excess rate as described above.

In preparation for the dissolution process, the reservoir 21 may be used as a convenient point for introducing fluid into the system. A desired volume of fluid may be poured into the reservoir 21 and the microcontroller 33 may be turned on. As the second fluid container 24 may be empty at this point, the fluid level sensor 31 may detect that the fluid level is low. The microcontroller 33 may receive an output from the sensor 31 and may switch on the recirculation pump 29. The recirculation pump 29 may begin to draw fluid from the reservoir 21 and transport it to the second fluid container 24, thereby filling it. The filling may stop when the sensor 31 detects a required level of fluid.

The provision of a recirculation pump 29 may also help ensure that the overall volume of fluid used for the dissolution process is small. This is because the recirculation pump 29 allows any fluid received from the flow cell 1 to be reused. The total volume of fluid may be between 10 and 5,000 millilitres, preferably about 250 millilitres.

As described above, it may also be desirable to control the temperature of the fluids during the dissolution process.

The temperature of the fluid may be monitored, such as by a temperature sensor. The temperature may be monitored at a point within the flow cell 1. It should be understood that the temperature may be monitored at any suitable location, including at the outlet 18 of the second cavity, or in a container collecting fluid flowing out through the outlet 18. Temperature measurements taken therein may be used to infer a temperature within the flow cell 1.

Fluid flow may be controlled such as by providing valves 25, 26 before the inlet to one or both of the first and second cavities 11, 12. Fluid flow may be stopped entirely as required such as by closing the valves 25, 26.

The amount of fluid in the reservoir 21 may be measured by placing a balance underneath it. By measuring the amount of fluid, a flow rate of fluid entering the reservoir 21 may be measured. Fluid flow may be controlled based on flow rate measurements, such as by controlling valves 25, 26.

In preparation for the dissolution process, the valves 25, 26 may also facilitate filling the various fluid-containing parts of the system, including the flow cell 1, the fluid containers 23, 24, and any tubes that are used. Initially, the valves 25, 26 may be open. As described above, the flow cell 1 may be positioned so that the outlets 13, 14 are at the highest points relative to the first and second cavities 11, 12. Fluid may be introduced. The fluid displaces any air and air bubbles in the flow cell 1, which may be allowed to float through inlets 13 and 14. The air may flow through valves 25 and 26. The air may flow through heat exchangers 27 and 28. The air may flow to containers 23 and 24. The air may leave the system at the open liquid surfaces in containers 23 and 24, which are open to the atmosphere. All tubes and containers and the flow cell 1 in the system may thus be filled with the fluid, and all air bubbles may thus be removed. The valves 25, 26 may then be shut. The flow cell 1 may be placed in a horizontal position such that the first cavity 11 is above the second cavity 12. The valves 25, 26 may be opened to allow fluid to begin flowing.

1. A dissolution device for measuring a dissolution rate of a test sample in a fluid, the device comprising:
a first cavity; and
a second cavity;
wherein each of the first and second cavities has a fluid inlet for connection to a fluid supply; and
wherein the device comprises an opening between the first and second cavities, and the device comprises a sample support configured to position the test sample across the opening.

2. The device of claim 1, wherein the sample support comprises a permeable support configured to support the test sample across the opening.

3. (canceled)

4. The device of claim 1, further comprising first and second fluid supplies, wherein the first and second cavities are in fluid communication respectively with the first and second fluid supplies via the respective fluid inlets, and wherein the first fluid supply is at a static pressure, measurable at a point within the first cavity and adjacent the test sample, that is equal to or higher than a static pressure of the second fluid supply, measurable at a point within the second cavity and adjacent the test sample.

5. The device of claim 4, wherein at least one of the first and second fluid supplies is configured to be substantially free of pressure pulsation.

6. The device of claim 4, wherein the first and second fluid supplies are configured to produce a substantially constant relative pressure therebetween.

7. The device of claim 4, wherein the static pressure of at least one of the first and second fluid supplies is provided by a hydrostatic pressure.

8. The device of claim 4, further comprising at least one of:
a first fluid container configured to feed fluid into the first fluid supply by gravity; and
a second fluid container configured to feed fluid into the second fluid supply by gravity.

9. The device of claim 8, further comprising a fluid level adjuster configured to adjust a vertical fluid level in at least one of the first and second fluid containers.

10. The device of claim 9, wherein the fluid level adjuster is configured to adjust the vertical fluid level by changing a vertical position of said at least one of the first and second fluid containers.

11. The device of claim 9, further comprising a sensor configured to monitor the vertical fluid level in at least one of the first and second fluid containers.

12. The device of claim 11, wherein the fluid level adjuster is configured to adjust the vertical fluid level in at least one of the first and second fluid containers based on an output of the sensor.

13-16. (canceled)

17. A method of measuring a dissolution rate of a test sample in a fluid, the method comprising the steps of:
providing a first supply of fluid in a first cavity;
providing a second supply of fluid in a second cavity, wherein an opening extends between the first and second cavities; and
positioning the test sample across the opening.

18. The method of claim 17, further comprising the step of providing a static pressure in the first cavity, measurable at a point within the first cavity and adjacent the test sample, that is higher than or equal to a static pressure within the second cavity, measurable at a point within the second cavity and adjacent the test sample.

19. The method of claim 17, wherein at least one of the first and second supplies of fluid is free of pressure pulsation.

20. The method of claim 17, wherein a relative pressure between the first and second supplies of fluid is substantially constant.

21. The method of claim 17, wherein a static pressure of at least one of the first and second supplies of fluid is provided by a hydrostatic pressure.

22. The method of claim 16, wherein:
the first supply of fluid is fed by gravity from a first fluid container; and/or
the second supply of fluid is fed by gravity from a second fluid container.

23. The method of claim 22, further comprising the step of adjusting a vertical fluid level in at least one of the first and second fluid containers to provide a required relative pressure between the first and second supplies of fluid.

24. The method of claim 23, further comprising the step of sensing the vertical fluid level in at least one of the first and second fluid containers.

25. The method of claim 24, wherein the vertical fluid level adjustment in at least one of the first and second fluid containers is based on the vertical fluid level sensing in at least one of the first and second fluid containers.

26-29. (canceled)
